(19) United States
(12) Patent Application Publication
(10) Pub. No.: US 2011/0270057 A1
(43) Pub. Date: Nov. 3, 2011

(54) DEVICE AND METHOD FOR DETECTION OF AN IN-VIVO PATHOLOGY

(76) Inventor: Amit Pascal, Haifa (IL)

(21) Appl. No.: 13/143,543

(22) PCT Filed: Jan. 7, 2010
(86) PCT No.: PCT/IL10/00015

§ 371 Date: Jul. 7, 2011

Related U.S. Application Data
(60) Provisional application No. 61/142,967, filed on Jan. 7, 2009.

(51) Int. Cl. A61B 5/1455

(52) U.S. Cl. 600/317

(57) ABSTRACT

An in-vivo sensing device for detecting in-vivo pathology may include an illumination source for illuminating light onto a tissue external to the device and an optical system for collecting fluorescent light emitted from the tissue onto a light sensor also provided within the device. A method of detecting in-vivo pathology by collecting fluorescent light emitted from a tissue is provided.

### FIELD OF THE INVENTION

The present invention relates to the field of detecting in-vivo pathologies. More specifically the present invention relates to a device and method for detecting fluorescent light emitted from in-vivo markers indicating a pathology.

### BACKGROUND OF THE INVENTION

Human and mammalian bodies enclose certain biological markers which may indicate in-vivo pathologies. When a pathology exists in-vivo, such biological markers, e.g. proteins, may be expressed and bound to the tissue. Early detection of these markers or biomarkers may lead to early detection of pathologies in the body which could lead to better treatment.

These markers may be detected by sensing an optical change which occurs due to binding of a binding agent (e.g. antibody or peptide) to a marker in-vivo, for example, detecting fluorescence at a given bandwidth, emitted from a binding agent bound to a marker in-vivo.

### SUMMARY OF THE INVENTION

Embodiments of the present invention provide a device and method for detecting fluorescent or other signals emitted from in-vivo markers. According to embodiments of the invention, fluorescent signals collected by the in-vivo device provided may be auto-fluorescence emitted from the tissue or may be fluorescence emitted from tissue which is labeled with fluorescent emitting molecules. According to embodiments of the present invention, fluorescent emitting molecules are typically attached to binding agents, which may bind to in-vivo markers. According to embodiments of the present invention, binding agents tagged with fluorescent emitting molecules may be administered into the body. The binding agents may have high affinity to specific in-vivo markers which indicate certain pathology searched for. The binding agents may bind to the in-vivo markers and a device may illuminate the tissue and its surroundings, while illuminating the fluorescent emitting molecules during its travel in-vivo. A device may illuminate in wavelengths that cause excitation to the fluorescent emitting molecules. The fluorescent emitting molecules emit fluorescent light in response to the excitation light illuminated from the device, and the device may then sense the emitted fluorescent light. Detecting fluorescent light by the device may indicate the presence of in-vivo markers which indicates the presence of pathology in the body. The in-vivo device may obtain fluorescent images along with images of the lumen it passes through. Depending on the device's optical design, the device may either illuminate the tissue at an alternating mode, i.e. the device may illuminate at white light alternatingly with illuminating at a wavelength that causes the tissue to excite, or the device may illuminate the tissue with the different wavelengths simultaneously. The in-vivo device may be a swallowable capsule, for example a capsule which may detect pathology in the gastrointestinal (GI) tract during its passage through the GI tract.

### BRIEF DESCRIPTION OF THE DRAWINGS

The present invention will be understood and appreciated more fully from the following detailed description taken in conjunction with the appended drawings in which:

FIG. 1 is a schematic illustration of in-vivo markers and an in-vivo device in accordance with the prior art;

FIG. 2 is a schematic illustration of an in-vivo sensing device in accordance with one embodiment of the present invention;

FIG. 3 is a schematic illustration of an in-vivo sensing device in accordance with another embodiment of the present invention;

FIG. 4 is a schematic illustration of an in-vivo sensing device in accordance with yet another embodiment of the present invention;

FIG. 5 is a schematic illustration of an in-vivo sensing device in accordance with another embodiment of the present invention; and

FIG. 6 is a flow-chart illustrating a method for detecting an in-vivo pathology in accordance with one embodiment of the invention.

It will be appreciated that for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as to not obscure the present invention.

When pathology is present in the body, in-vivo markers may be expressed. The in-vivo markers may be attached to the tissue where the pathology is present.

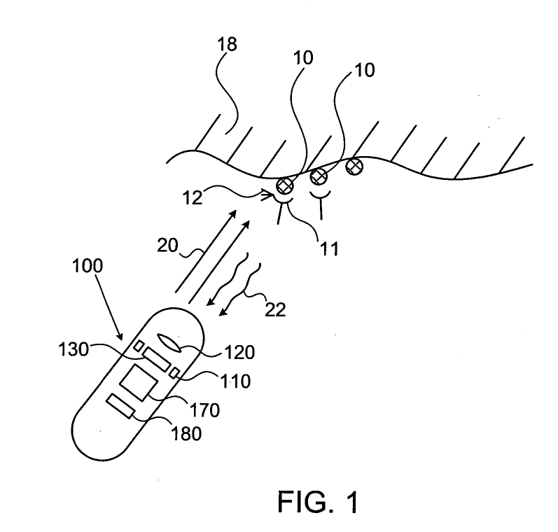

Reference is now made to FIG. 1, which is a schematic illustration of in-vivo markers in accordance with an embodiment of the invention. As depicted in FIG. 1, in-vivo markers 10 may be attached onto tissue 18. When the tissue is illuminated at a wavelength 20 which may cause excitation to the in-vivo markers, auto-fluorescence 22 may be detected. The tissue may be illuminated by an in-vivo device 100 which may include at least one illumination source 110, an optical system 120 (e.g., including a lens) and a light sensor 130. The illumination source 110 from in-vivo device 100 may illuminate the tissue and the optical system 120 may collect the fluorescent signal reflected by the tissue onto the light sensor 130 within device 100. In other embodiments, binding agents 11 which have high affinity to the in-vivo markers may be administered to a patient for example orally, systemically or by an enema. Binding agents 11 may have attached thereon a fluorescent emitting molecule 12. When in-vivo device 100 illuminates at a wavelength 20 which causes excitation to the fluorescent emitting molecule 12, fluorescent light 22 is emitted from molecules 12 which may then be sensed by the light sensor 130.

Non-limiting examples of in-vivo markers 10 which are expressed in the colon tissue, and indicate on cancer are: S100A8 Protein S100-A8, S100A9 Protein S100-A9, CEACAM5 Protein (CEA), OLFM4 Olfactomedin-4 precursor, Underglycosilated Mucin-1 (uMUC-1), Matrix metalloproteinase 7 (MMP-7), Cathepsins (especially Cathepsin B), LTF Lactotransferrin precursor, MPO Isoform H7 of Myeloperoxidase precursor, TNC Isoform 1 of Tenascin precursor, and Epidermal growth factor receptor EGFR.

Non-limiting examples of fluorescent emitting molecules 12 are: MMPSense 680 activatable fluorescence agent, MMPSense 750 FAST activatable fluorescence agent, ProSense 680 activatable fluorescence agent, and ProSense 750 activatable fluorescence agent commercially available by VisEn. Other examples of fluorescent emitting molecules 12 are 2-DG Optical Probe, EGF Optical Probe, and RGD Optical Probe commercially available by LI-COR® Biosciences. In other embodiments, molecules 12 need not be fluorescent emitting molecules but may rather be gold nanoparticles.

In some embodiments, in-vivo device 100 may be a swallowable capsule and may detect pathologies within the GI tract during its passage through it. In some embodiments, in-vivo device 100 may include a power source 170 which may be at least one battery. Device 100 may include a transmitter 180 including an antenna which may transmit the images of fluorescent light 22 acquired by light sensor 130, to an external receiver and/or a display system (not shown).

Device 100 may be or may include an autonomous swallowable capsule, but device 100 may have other shapes and need not be swallowable or autonomous. Embodiments of device 100 are typically autonomous, and are typically self-contained. For example, device 100 may be a capsule or other unit where all the components including, for example, power components are substantially contained within a container, housing or shell, and where device 100 does not require any wires or cables to, for example, receive power or transmit information. Device 100 may communicate with an external receiving and display system to provide display of data, control, or other functions. For example, in an autonomous system power may be provided by an internal battery or a wireless receiving system. Other embodiments may have other configurations and capabilities. For example, components may be distributed over multiple sites or units. Control information may be received from an external source

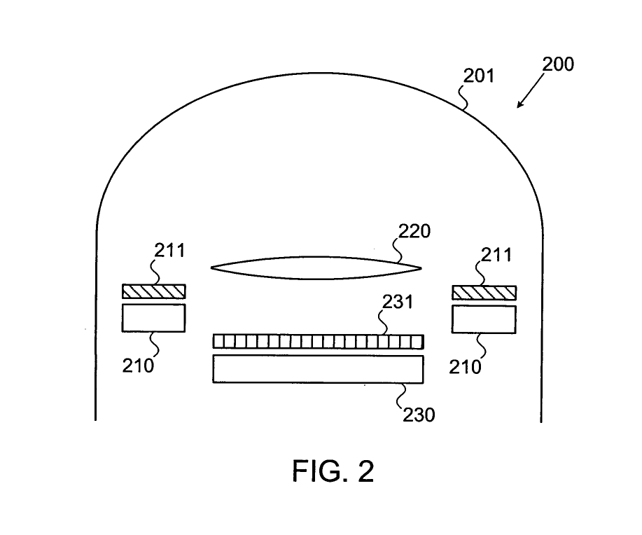

Reference is now made to FIG. 2 which is a schematic illustration of an in-vivo sensing device in accordance with one embodiment of the present invention. FIG. 2 depicts a fluorescent detecting layout of an in-vivo sensing device 200 which may be similar to in-vivo device 100 shown in FIG. 1. FIG. 2 depicts a dome or window 201, behind which are positioned (on the concave side of window 201) and through which operate illumination sources 210, optical system 220 and light sensor 230. Illumination sources 210, optical system 220 and light sensor 230 are positioned behind (and/or to some extent within) dome or window 201, but within the device 100 body or housing. E.g., illumination is provided via window 201, images and light are received via window 201, and light sensor, such as light sensor 230, receive light and/or images via window 201. Typically there are at least two illumination sources within device 200. In some embodiments, illumination sources 210 may be arranged in a ring configuration, e.g. a ring of light emitting diodes (LEDs). In some embodiments, illumination sources 210 may be white LEDs, a vertical cavity surface emitting laser (VCSEL) or monochrome LEDs. In order to illuminate tissue or in-vivo markers attached to a tissue and thereby cause the tissue or markers to emit fluorescent light, illumination sources 210 may have attached thereon excitation filters 211. Excitation filters 211 are cleaning filters that may only allow passage of illumination at a wavelength which causes excitation to either a tissue, or to in-vivo markers or to fluorescent emitting molecules attached to in-vivo markers as described in FIG. 1, such to avoid illumination sources 210 from illuminating light in other wavelengths, e.g. wavelengths which a light sensor that includes an emission filter might detect. In some embodiments, light sensor 230 may include a filter that only enables passage of wavelength correlating to fluorescent light emitted from the tissue. In some embodiments, if illumination sources 210 lacks cleaning filters, some of the light reflected from the tissue may include light in excitation wavelengths that are close to the emission wavelength i.e. such light may pass through the emission filter. This might create a screening effect on the image detected by light sensor 230 In some embodiments, excitation filters 211 may be short pass filters or band pass filters that only enable passage of illumination at a wavelength which may cause excitation.

In some embodiments, following excitation of the tissue or in-vivo markers or fluorescent emitting molecules, fluorescent light may be emitted from the tissue or in-vivo marker or from a fluorescent emitting molecule. The fluorescent light emitted may be collected and focused by optical system 220 onto light sensor 230. In some embodiments, optical system 220 may comprise one or more lenses for collecting fluorescent light emitted from the tissue, markers or fluorescent emitting molecule.

In some embodiments, light sensor 230 may have attached thereon or disposed thereon (e.g., placed on or near) an emission filter 231. Emission filter 231 blocks light at a wavelength of excitation and allows passage of light at a wavelength of emission from the tissue or markers or fluorescent emitting molecule. In some embodiments, light sensor 230 may be a black and white imager, e.g. silicon with no color filters, such that areas emitting fluorescent signals may be viewed as white areas and areas, from which no fluorescent signal was emitted may be viewed as black areas. In between the black and white areas there may be grey level areas which indicate low signals of fluorescence emitted from the background tissue. The type of excitation filter 211 that may be used in order to pass light that causes excitation may be chosen per the fluorescent emitting molecule it is to excite. However, healthy tissue surrounding the tissue with a pathology may emit auto-fluorescence at different levels of intensity, typically low intensity compared to the tissue expressing in-vivo markers indicating the pathology. The background tissue emitting fluorescence at a low intensity may be viewed as grey areas on the light sensor 230, such that the image sensed by light sensor 230 may be a grey scale image.

In some embodiments, emission filter 231 may be positioned between light sensor 230 and optical system 220. Emission filter 231 may be attached onto or disposed on (e.g., placed on or near) light sensor 230 and may cover the surface of light sensor 230, such that light reflected from the tissue may be collected and focused by optical system 220 onto light sensor 230 while first being filtered by emission filter 231. In other embodiments, when optical system 220 includes more than one lens, emission filter 231 may be positioned in between the lenses of optical system 220. In other embodiments, emission filter 231 may be positioned between optical system 220 and window 201, such that light reflected from the tissue and which passed through window 201, is first filtered and only then focused by optical system 220 onto light sensor 230. In some embodiments, dome or window 201 may be dome shaped (e.g., substantially hemispherical). In some embodiments, in-vivo sensing device 200 may be a swallowable capsule.

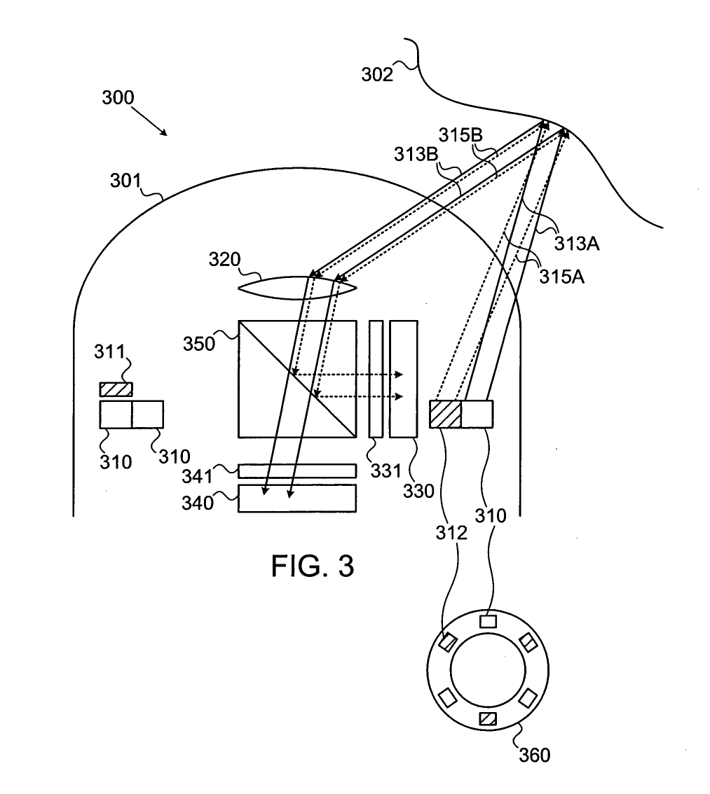

Reference is now made to FIG. 3 which is a schematic illustration of an in-vivo sensing device in accordance with another embodiment of the present invention. FIG. 3 depicts a fluorescent detecting layout of an in-vivo sensing device 300. In some embodiments, in-vivo sensing device 300 may comprise a dome or window 301 through which illumination from illumination sources 310 and/or 312 illuminate tissue 302 (or markers attached onto it). In some embodiments, illumination source 310 may be a white light illumination source, e.g. white LED, while illumination source 312 may illuminate at a wavelength which may cause excitation to tissue 302, e.g. at a wavelength between the range of UV and near infra red (IR). Typically, illumination source 312 illuminates at a wavelength which causes excitation to a tagged binding agent bound to an in-vivo marker attached to tissue 302. In some embodiments, the binding agent which has high affinity to a specific in-vivo marker may be tagged with a fluorescent emitting molecule, the latter emitting fluorescence when illuminated at a wavelength which causes excitation.

In other embodiments, there may be at least two illumination sources 310 which illuminate white light, however, one of the illumination sources 310 may comprise an excitation filter 311 which is a cleaning filter that may only allow passage of light at a wavelength which causes excitation to the tagged binding agent-marker complex. In some embodiments, the illumination sources 310 and 312 (or two illumination sources 310, where one comprises filter 311) are attached onto a ring shaped substrate 360, which may be placed on a printed circuit board (PCB) within the device 300. Typically, the illumination sources 310 and 312 (or illumination sources 310, and 310 comprising filter 311) are positioned on ring 360 in an alternating configuration, such that there is one illumination source of one kind positioned on either side of the illumination source of the other kind, and vice versa.

Behind window 301 and operating through it (e.g., collecting light via it), may further be positioned an optical system 320 which collects illumination reflected from the tissue 302. In some embodiments, positioned behind window 301 may be a light sensor 330 which may be designed to sense fluorescent light emitted from the tissue 302 and collected by optical system 320. In some embodiments, light sensor 330 may have attached to it or disposed on it (e.g., placed on or near) an emission filter 331. In some embodiments, emission filter 331 may only enable fluorescent light emitted from the tissue to pass through it. In some embodiments, light sensor 330 may be a black and white imager.

In some embodiments, there may be an additional light sensor 340, typically a color or red-green-blue (RGB) imager, which may be designed to sense white light reflected from tissue 302, or to sense light reflected from tissue when a substantially white-light source is directed at the tissue, in order to create a color image as well as a fluorescent map of tissue 302, which is created by light sensor 330. Light sensor 340 may be capable of detecting color images, and may receive images created when white light is directed at tissue. In some embodiments, light sensor 340 may be a charge coupled device (CCD) imager or a complimentary metal oxide semiconductor (CMOS) imager, typically a color imager. In some embodiments, light sensor 340 comprises a notch filter 341. In some embodiments, notch filter 341 may block illumination at a wavelength of excitation, i.e. direct light illuminated from illumination source 312 or from illumination source 310 which comprises the excitation filter 311. A notch filter 341 is typically used when the excitation wavelength needed in order to cause excitation to the tagged tissue 302 is near the white light wavelengths. In other embodiments, when the excitation wavelength is longer than white light, the filter 341 attached onto or associated with light sensor 340 may be a short-pass filter.

According to embodiments of the present invention, light sensor 340 is oriented to face perpendicular to (e.g., the plane of the imager may be substantially perpendicular to the longitudinal axis of symmetry of the device), to face substantially perpendicular to, or to face not parallel to, a direction of movement of device 300. In some embodiments, light sensor 330 is positioned perpendicular or substantially perpendicular to, or not in a parallel plane with, light sensor 340 and parallel to or substantially parallel to the direction of movement of device 300. In some embodiments, light sensor 330 extends in a plane perpendicular to or substantially perpendicular to the plane light sensor 340 is in. In some embodiments, between the two light sensors 330 and 340 is positioned a dichroic filter (or dichroic mirror) 350. The dichroic filter 350 is positioned behind the window 301 through which light is illuminated onto the tissue and through which light is reflected from the tissue onto the light sensors 330 and 340. Dichroic filter 350 is oriented such that it is positioned between light sensor 330 and light sensor 340, which are also perpendicular or substantially perpendicular to one another. In some embodiments, optical system 320 collects light reflected from the tissue 302 after tissue 302 is illuminated by illumination sources 310 and 312. The dichroic filter 350 is designed to reflect and/or transmit the reflected light which passes through optical system 320, onto the respective light sensor. For example, illumination rays 313a illuminated from illumination source 310, which illuminates white light, reach the tissue 302. Illumination rays 313b are then reflected from the tissue 302 and collected by optical system 320 onto dichroic filter 350. Dichroic filter 350 may then transmit light rays 313b to filter 341. Light rays 313b may be filtered by filter 341 and projected onto light sensor 340, which is designed to sense white light reflected from the tissue 302, excluding excitation light. Filter 341 may be, for example, a notch filter or a short-pass filter which may block illumination at a wavelength of excitation.

According to some embodiments, at the same time that white light 313b is collected and sensed by light sensor 340, excitation light may be sensed by another sensor. In some embodiments, excitation light rays 315a are illuminated from illumination source 312 (or from illumination source 310 having attached thereon or disposed thereon (e.g., placed on or near) an excitation filter 311) onto tissue 302. In some embodiments, tissue 302 may be tagged with fluorescent emitting molecules, which are attached to binding agents that bind to pathology related in-vivo markers. Light rays 315a may cause excitation to the typically tagged tissue 302. The tissue 302 may emit fluorescent light rays 315b when excited or when the fluorescent emitting molecules attached to the tissue are excited. Fluorescent light rays 315b may be collected by optical system 320 onto dichroic filter 350. Dichroic filter 350 may then reflect fluorescent light rays 315b to filter 331. Light rays 315b may be filtered by filter 331 and projected onto light sensor 330, which is designed to sense fluorescent light emitted from the tissue 302. Filter 331 may be an emission filter which only enables passage of fluorescent light at a wavelength of emission from the tagged tissue 302. In some embodiments, fluorescent emission is between red and near IR wavelengths. Fluorescent light rays 315b when sensed by light sensor 330, may create a map of fluorescent emission from the tissue 302.

In some embodiments, the dichroic filter enables device 300 to illuminate tissue 302 with white light and with excitation light, simultaneously. In other embodiments, illumination 313a from illumination source 310 and illumination 315a from illumination source 312 may be illuminated onto tissue 302 in an alternating mode.

According to embodiments of the present invention, illuminating in alternating mode refers to alternating the illuminating periods of different illumination sources. For example, illuminating the tissue with a second illumination source for a certain period of time subsequent to illuminating the tissue with a first illumination source, and then illuminating with the first illumination source again, and so forth. Different illuminating periods may be used for each illumination source. In some embodiments, illuminating with the first and second illumination sources is sequential, and occurs with a brief separation of time. In some embodiments there may be more than two illumination sources illuminating in an alternating mode.

As with the other swallowable devices discussed herein, in some embodiments, window 301 may be dome shaped. In some embodiments, device 300 may be a swallowable capsule.

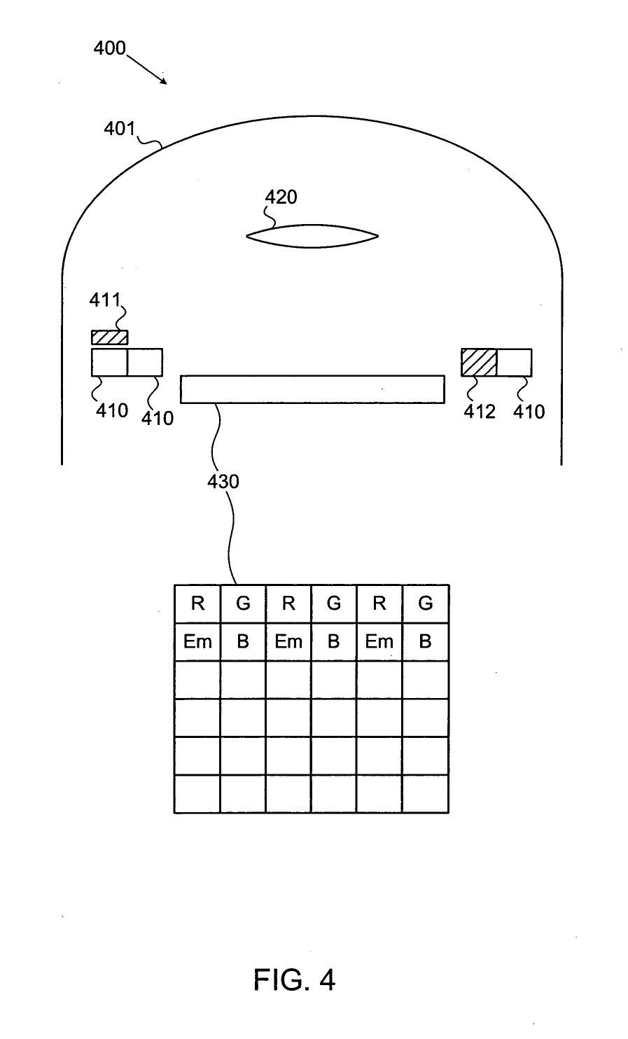

Reference is now made to FIG. 4 which is a schematic illustration of an in-vivo sensing device in accordance with yet another embodiment of the present invention.

FIG. 4 depicts a fluorescent detecting layout of an in-vivo sensing device 400. In some embodiments, in-vivo sensing device 400 may include a dome or window 401 through which illumination from illumination sources 410 and/or 412 illuminate a tissue (or markers attached onto it). In some embodiments, illumination source 410 may be a white light illumination source, e.g. white LED, while illumination source 412 may illuminate at a wavelength which may cause excitation to the tissue, e.g. between the range of UV and near infra red (IR). Typically, illumination source 412 illuminates at a wavelength which cause excitation to a tagged binding agent bound to an in-vivo marker attached to a tissue. In some embodiments, the binding agent which has high affinity to a specific in-vivo marker may be tagged with a fluorescent emitting molecule, the latter emitting fluorescence when illuminated at a wavelength which causes it to excite.

In other embodiments, there may be at least two illumination sources 410 which illuminate white light, however one of the illumination sources 410 may have attached or associated with it an excitation filter 411 which may only allow passage of illumination at a wavelength which cause excitation to the tagged binding agent-marker complex. In some embodiments, the illumination sources 410 and 412 are attached onto a ring shaped substrate, which is positioned on a PCB within the device 400, as ring 360 shown in FIG. 3.

In some embodiments, device 400 may comprise an optical system 420 (e.g. including one or more lenses or other structures) positioned behind window 401, which may collect reflected light from the tagged tissue onto light sensor 430 which is also positioned behind window 401. According to some embodiments, light sensor 430 may be a CCD or CMOS imager. In some embodiments, light sensor 430 may include a color filter array (CFA) such that different pixels of the light sensor pixel array include different color filters. The CFA may include four types of color filters, e.g. red sensitive elements, blue sensitive elements, green sensitive elements and an element sensitive to fluorescent light at a wavelength of emission from the tagged tissue. Typically, the CFA is arranged such that the color sensitive elements alternate in groups of four, for example as shown in FIG. 4, a red sensitive element R may be positioned in a first position, and to its right, a green sensitive element G may be positioned. Below the red sensitive element R and diagonally to the green sensitive element G, may be an emission sensitive element Em, and to its right, there may be a blue sensitive element B, such that all four colors create a small four elements array. Such arrays of the group of four color sensitive elements may be arranged side by side, in order to create a complete CFA attached onto light sensor 430. Other specific arrangements and other numbers of elements may be used.

According to an embodiment shown in FIG. 4, there is no filter for blocking excitation light positioned between optical system 420 and light sensor 430, or positioned between optical system 420 and window 401, since the filters needed are placed directly onto light sensor 430 in the form of a CFA.

In some embodiments, the image that the light sensor 430 may obtain is a color image (from white light reflected from a target) along with a fluorescent emission light image. Since the CFA on light sensor 430 may be built from four different color sensitive elements (e.g. R, G, B, Em, as disclosed above), or another number of elements, color information of the imaged tissue may be sensed along with sensed fluorescent emission information, since the group of four image about the same area of the tissue. In some embodiments, the color image and the fluorescent image may be shown on a display external to device 400, on the same image, yet in other embodiments, the RGB color image and the fluorescent image may be shown side by side. When the RGB color image is overlaid with the fluorescent image, the location of the pathology in-vivo may be more visible.

In an exemplary embodiment, device 400 may be a swallowable autonomous capsule that passes along a patient's GI tract. While device 400 traverses a patient's GI tract, device 400 transmits image and possibly other data to components located outside the patient's body which receive and process the data. Preferably, two images (a color image and a fluorescent image) using different illumination sources 410 and 412 (or 410 and 410 with cleaning filter 411) are captured 20 milliseconds apart, stored in the device 400, and transmitted as one burst of information; one second later another two images are captured. Other time differentials may be used. The two images may be transmitted as two separate images or, may be processed and interlaced or combined into one image before transmission. The images may be combined by interleaving by bit or by pixel before transmission, or otherwise interleaved or combined. In other embodiments, the images may be multiplexed through known methods. In yet other embodiments, other rates of imaging and other timing schemes may be used.

In some embodiments, illumination sources 410 and 412, or illumination sources 410 and illumination sources 410 comprising an excitation filter 411 may illuminate simultaneously. In other embodiments, illumination sources 410 and 412, or 410 and 410 with filter 411 may illuminate alternatingly. For example, illumination source 410 may illuminate at a certain frequency for a predefined period and when that predefined period elapses, illumination source 412 (or 410 with filter 411) may illuminate at another frequency (either at the same frequency as the first illumination source, or at a different frequency) for another predetermined period and so on, one illumination source operating subsequent to the other.

However, in some embodiments, during the operation of illumination sources that illuminate at a wavelength that cause excitation to the tissue (illumination source 412 or illumination sources 410 which comprise an excitation filter), the RGB color sensitive elements placed on light sensor 430 may sense the excitation light and thus experience full well saturation. Although the illumination sources in device 400 may operate in an alternating mode, e.g. subsequent to one another, if the RGB color sensitive elements sense excitation light in addition to sensing RGB light, their wells may be saturated and thus may lose their ability to accommodate additional charge. The additional charge may then spread into neighboring pixels, causing them to either report erroneous values or to saturate as well. This spread of charge may appear as a white streak or blob in the image.

There may be a few solutions for overcoming such possible white streaks in the images obtained by light sensor 430. One solution may be to use different well capacities. For example, the RGB color sensitive elements' well capacity may be larger than the Em color sensitive well's capacity, such that it would take longer for the RGB wells to saturate. Another solution may be to continuously sample the RGB wells during the operation of excitation illumination sources, such that the RGB wells will not be saturated. Yet another solution may be adding a notch filter over light sensor 430. The notch filter may block excitation light alone, such that illumination sources 410, 412 or 410 with excitation filter 411 may either operate simultaneously or in alternating mode.

According to some embodiments, notch filter (not shown) for blocking excitation light may be positioned between optical system 420 and light sensor 430, so that light reflected from the tissue is focused onto the notch filter, filtered in order to block excitation light and then be projected onto light sensor 430. In other embodiments, the notch filter may be positioned between optical system 420 and window 401, so that light reflected from the tissue is first filtered and only then focused onto light sensor 430.

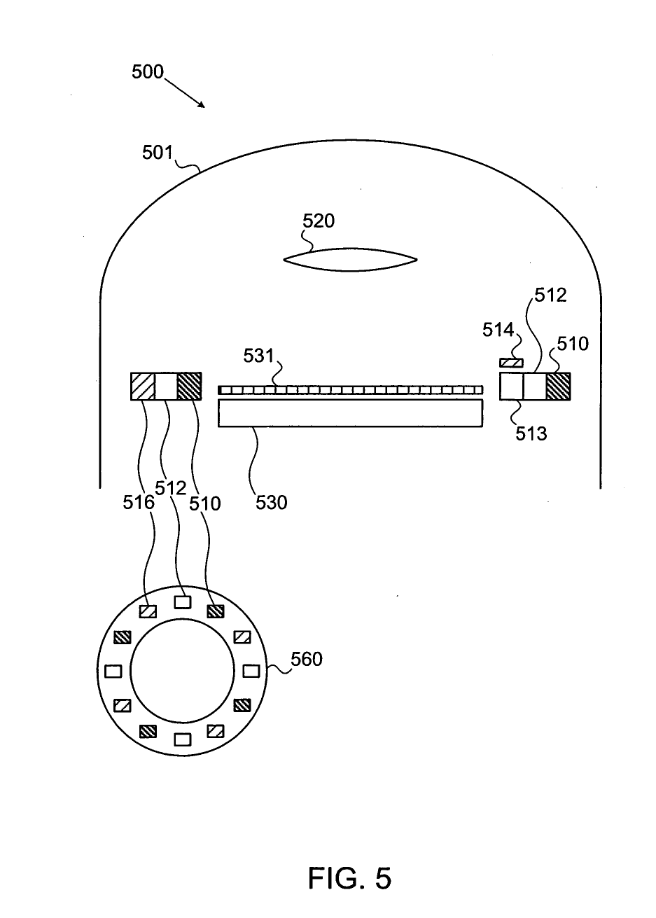

Reference is now made to FIG. 5 which is a schematic illustration of an in-vivo sensing device in accordance with another embodiment of the present invention. In-vivo sensing device 500 may include dome or window 501 behind which (or partially within) are positioned at least one blue illumination source 510 which illuminates light at a wavelength within the blue spectrum, and at least one green illumination source 512 which illuminates light at a wavelength within the green spectrum. In other embodiments, other combinations of illumination sources may be used. Device 500 typically comprises at least two different illumination sources selected from the blue, green and red spectrums. At least two different red, green or blue illumination sources may be used in order to obtain an image similar to an image obtained with white light. In some embodiments, the at least two colored images may be combined using image processing methods in order to obtain an image similar to a white light image. In other embodiments, device 500 may include illumination sources in all three spectrums, e.g. at least three illumination sources; one illuminating within the red spectrum, one within the green spectrum and one with the blue spectrum. Device 500 may further include at least one excitation light source 516, which illuminates light at a wavelength that may cause excitation to a tissue external to device 500. The specific excitation wavelength in which illumination source 516 illuminates the tissue may be chosen by the specific in-vivo markers attached onto the tissue to be excited. Typically, the in-vivo markers bind to a specific binding agent administered in-vivo, which has high affinity to the markers and which may be tagged with a fluorescent emitting molecule which may emit fluorescent light when illuminated in a proper wavelength that causes excitation. Therefore, the wavelength of excitation illumination source 516 is such that it is to cause excitation to the fluorescent emitting molecule attached to the binding agent-marker complex. In other embodiments, illumination source 513, which may be for example, a white LED, may have attached thereon or associated therewith an excitation filter 514 which may allow passage of illumination only at a wavelength which may cause excitation to the tagged tissue.

According to some embodiments, illumination sources 510, 512 and 516 (or 513 with filter 514) may be positioned on a ring shaped substrate which may be placed on a PCB within device 500. In some embodiments, the arrangement of the illumination sources 510, 512 and 516 (or 513 with filter 514) may be in alternating configuration as shown in ring 560. For example, illumination source 510 is positioned next to illumination source 512, which is positioned next to illumination source 516, which is placed besides illumination source 510 and so on, while all sources are attached onto ring 560. In some embodiments, the illumination sources may operate simultaneously, such that the tagged tissue is illuminated in blue, green (and red) and excitation light, all at once. In other embodiments, the illumination sources may illuminate the tagged tissue alternatingly, such that the tissue may first be illuminated in blue light, for example, then in green light, then in red light, and then in excitation light. Other orders may be used.

In some embodiments, device 500 may include optical system 520, which may comprise one or more lenses or other components. In some embodiments, there may be a light sensor 530, which may be a CCD or CMOS RGB imager. Light sensor 530 may include a notch filter 531 which may block illumination at a wavelength of excitation, such that the only light which may pass through notch filter 531 is light reflected from the tagged tissue. Experiments show that some types of excitation light and emission light are found in the red spectrum. Therefore, if notch filter 531 blocks excitation light, the red sensitive pixels in light sensor 530 will actually sense light at a wavelength of emission, e.g., fluorescent light emitted from the tagged tissue. The blue and green sensitive pixels in light sensor 530 sense the reflected blue and green illumination, such that from the light sensed by light sensor 530 it would be possible to create a blue-green image of the tagged tissue overlaid with a fluorescent map of the tissue. In some embodiments, the blue-green image may be shown on a display external to device 500 along side with the fluorescent image. In other embodiments, the color image may be combined/overlaid with the fluorescent image, such that they are shown as one image.

According to other embodiments, light sensor 530 may be a black and white imager, e.g. silicon with no color filters, and thus illumination sources 510, 512 and 516 may operate in an alternating mode, e.g. one illumination source (or set of sources sharing a wavelength) illuminates the tissue subsequent to another illumination source.

According to some embodiments, device 500 may be a swallowable capsule. In some embodiments, window 501 may be dome shaped. According to other embodiments, device 500 or other devices shown herein may be implemented in an endoscope. For example, an endoscope may include four illumination sources, e.g. four colored LEDs; one red, one green, one blue and one with a filter that enables illuminating at a wavelength that causes excitation to the tissue or to the fluorescent emitting molecules attached to the tissue. In other embodiments, there may be only three illumination sources; two selected in any combination from red, green, and blue LEDs and one that illuminates in excitation wavelength. In other embodiments there may be more than one set of three illumination sources (or four illumination sources). The illumination sources in the endoscope need not be arranged in a ring shape, but rather be arranged in a row, half a circle or any other configuration. The endoscope may further comprise a light sensor, e.g. a black and white imager, which requires adding a notch filter and illuminating in an alternating mode.

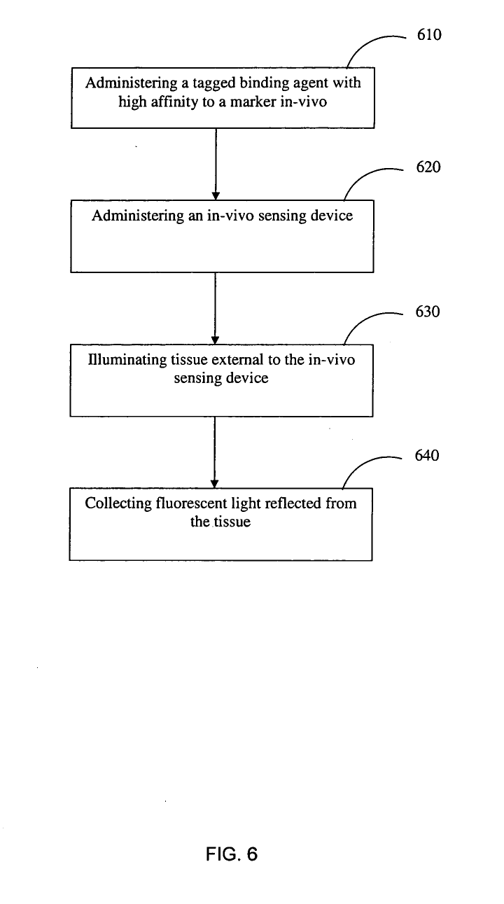

Reference is now made to FIG. 6 which is a flow-chart illustrating a method for detecting in-vivo pathology in accordance with one embodiment of the invention. According to some embodiments, the method may include administering to a patient an in-vivo sensing device comprising an illumination source, an optical system and a light sensor (block 620). For example, the device may be inserted into a patient by, for example, swallowing. The in-vivo sensing device may be for example devices 200, 300, 400 or 500, or another suitable device. The method may further comprise illuminating in-vivo tissue which is external to the in-vivo sensing device (block 630), and collecting fluorescent light reflected from the tissue onto the light sensor by using the optical system (block 640). In some embodiments, the fluorescence may not be fluorescence emitted from the tissue, or from markers attached onto the tissue, which are present when there is pathology in-vivo, but rather fluorescence from a binding agent with high affinity to specific in-vivo markers prior ingested by or inserted into the patient prior to administering the in-vivo sensing device (block 610). In some embodiments, the patient may ingest the binding agents by swallowing it, injecting it or through an enema. After the patient has inserted the binding agents into the body, the patient may then insert the in-vivo sensing device into the body. The in-vivo sensing device may be capsule shaped, but may have other shapes which allow easy and comfortable insertion in-vivo.

Typically, the binding agent may have attached a fluorescent emitting molecule, such that when the in-vivo sensing device illuminates the complex of marker-binding agent-fluorescent emitting molecule, the molecule, when illuminated in an excitation wavelength is excited and thus emits fluorescent light. The emitted fluorescent light may then be collected by the optical system within the in-vivo sensing device onto the light sensor within the device.

According to some embodiments, the method may include collecting light reflected from the tissue, which is light at a wavelength different than the fluorescent light reflected from the tissue. In some embodiments, the optical system may collect all or almost all light reflected from the tagged tissue, and focus it onto the light sensor within the device. In some embodiments, the light sensor may be designed so as to sense light at various wavelengths.

In some embodiments the device may comprise at least one illumination source for illuminating white light and at least one illumination source for illuminating light which causes excitation to in-vivo tissue. In some embodiments, the method may then comprise the steps of collecting white light reflected from the tissue, collecting fluorescent light emitted from the tissue, and creating a color image of the tissue along with a fluorescent image. A color image of the tagged tissue may be created along with a fluorescent image mapping the location of the in-vivo markers from which fluorescent signal were emitted.

Other operations or series of operations may be performed.

It will be appreciated that the present invention is not limited to what has been particularly shown and described hereinabove. Rather the scope of the present invention is defined only by the claims which follow.

1. An in-vivo sensing device for detecting an in-vivo pathology, said device comprising:
a housing; and
a dome, wherein said housing comprises:
an excitation illumination source for illuminating light at a wavelength which causes excitation to a tissue external to said device;
an optical system; and
a light sensor,
wherein said optical system is for collecting fluorescent light reflected from the tissue onto said light sensor, and
wherein said illumination source, said light sensor and said optical system are positioned on the concave side of said dome.

2. The device according to claim 1, wherein said at least one illumination source comprises an excitation filter allowing to pass only illumination at a wavelength which causes excitation to the tissue.

3. The device of claim 1, comprising an emission filter allowing to pass only fluorescent illumination emitted from the tissue, said emission filter positioned between said light sensor and said dome.

4. The device according to claim 3, wherein said emission filter is positioned between said light sensor and said optical system.

5. The device according to claim 3, wherein said emission filter is positioned between said optical system and said dome.

6. The device of claim 2, wherein said excitation filter is selected from the group consisting of a short pass filter, a band pass filter and a combination thereof.

7. The device of claim 1, wherein said at least one illumination source is selected from the group consisting of: a white LED, a monochromatic LED, and a VCSEL.

8. The in-vivo sensing device according to claim 1, wherein said device comprises: a white light illumination source for illuminating white light onto a tissue external to said device; and wherein said light sensor comprises: a color filter array (CFA), said CFA comprising red sensitive elements, blue sensitive elements, green sensitive elements and elements sensitive to a fluorescent illumination at a wavelength of emission from the tissue.

9. The in-vivo device according to claim 8, further comprising an excitation filter for passing through illumination at a wavelength which causes excitation to the tissue, wherein said excitation filter is disposed on said at least one excitation illumination source for illuminating light at a wavelength which cause excitation to the tissue.

10. The in-vivo sensing device of claim 8, wherein said at least one white light illumination source and said at least one excitation illumination source illuminate alternatingly.

11. The in-vivo sensing device according to claim 1, wherein said device comprises: at least two different illumination sources selected from: a blue illumination source for illuminating blue light onto said tissue, a green illumination source for illuminating green light onto said tissue and a red illumination source for illuminating red light onto said tissue.

12. The in-vivo sensing device according to claim 11, further comprising a notch filter for passing through illumination at a wavelength other than an excitation wavelength, wherein said notch filter is disposed on light sensor.

13. The in-vivo sensing device of claim 11, wherein said at least two illumination sources selected from said blue, green or red illumination sources and said at least one excitation illumination source illuminate alternatingly.

14. The in-vivo sensing device of claim 11, wherein said light sensor is a black and white imager.

15. (canceled)

16. An in-vivo sensing device for detecting in-vivo pathology, said device comprising:
a housing; and
a dome, wherein said housing comprises:
a white light illumination source for illuminating white light onto a tissue external to said device;
an excitation illumination source for illuminating light at a wavelength which causes excitation to the tissue;
a first light sensor for sensing white light;
a second light sensor for sensing fluorescent light emitted from the tissue, wherein said first light sensor is oriented perpendicularly to said second light sensor;
an optical system; and
a dichroic filter positioned between said first light sensor and said second light sensor,
wherein said optical system is for collecting illumination reflected from the tissue onto said dichroic filter, and wherein said dichroic filter is for transmitting reflected white light on said first light sensor and for reflecting emitted fluorescent light onto said second light sensor,
and
wherein said at least one white light illumination source, said at least one excitation illumination source, said first and second light sensors, said optical system and said dichroic filter are positioned behind a concave side of said dome.

17. The in-vivo device according to claim 16, further comprising an excitation filter allowing to pass only illumination at a wavelength which causes excitation to the tissue, wherein said excitation filter is disposed on said at least one excitation illumination source for illuminating light at a wavelength which causes excitation to the tissue.

18. The in-vivo sensing device of claim 16, further comprising a short pass filter for passing through illumination at a wavelength other than wavelength which causes excitation to the tissue, wherein said short pass filter is disposed on the first light sensor for sensing white light.

19. The in-vivo device of claim 16, further comprising a notch filter for passing through illumination at a wavelength other than a wavelength which causes excitation to the tissue, wherein said notch filter is disposed on first light sensor.

20. The in-vivo sensing device of claim 16, further comprising an emission filter for passing through fluorescent illumination emitted from the tissue, wherein said emission filter is disposed on the second light sensor.

21. The in-vivo sensing device of claim 16, wherein said at least one white light illumination source and said at least one excitation illumination source illuminate alternatingly.

22. (canceled)

23. (canceled)

24. (canceled)

25. (canceled)
